(19) United States
(12) Patent Application Publication
(10) Pub. No.: US 2015/0335467 A1
(43) Pub. Date: Nov. 26, 2015

(54) PATIENT WARMING SYSTEM CONNECTION DEVICE

(71) Applicant: CareFusion 2200, Inc., San Diego, CA (US)

(72) Inventors: Christopher M. Varga, Laguna Hills, CA (US); Jason Anthony Mohr, Fontana, CA (US); Benjamin T. Krupp, Wyoming, OH (US)

(73) Assignee: CareFusion 2200, Inc., San Diego, CA (US)

(21) Appl. No.: 14/286,641

(22) Filed: May 23, 2014

(51) Int. Cl. A61F 7/00

(52) U.S. Cl. CPC A61F 7/0085; A61F 2007/0045; A61F 2007/0054

(57) ABSTRACT

A connection device for a patient warming system is provided. The device includes a first connector port and a second connector port that each includes a wall that circumferentially defines a longitudinal channel. The first and second connection ports are each configured to receive a tubing that leads to a patient warming device. A self-sealing valve is aligned in each of the longitudinal channels to engage an inlet and outlet port of a control unit of the patient warming system. A planar base member of the connection device joins the second end of the first connector port generally parallel to the second end of second connector port. The connection device also includes a flexure member with a first catch mechanism that releasably secures the connection device to the control unit of the patient warming system.

### TECHNICAL FIELD

Embodiments disclosed in the present application relate generally to connection devices for coupling tubing and other components of patient warming systems.

### BACKGROUND

During surgical procedures, patients may be placed under anesthesia. As a result, the body's natural thermoregulatory mechanisms may be affected and systemic vasodilation may occur. Systemic dilation counteracts the body's natural heat retention mechanism and allows body heat to flow down a concentration gradient to the extremities, where heat is lost to the environment. As a result, the patient is at risk of perioperative hypothermia. Medical complications may result from perioperative hypothermia and may include peri-operative and post-operative complications, including for example, increased wound infection rates, metabolic acidosis, respiratory distress, cardiovascular effects, surgical bleeding, and increased risk of mortality. Therefore, a need exists for patient warming systems that actively warm the patient to maintain normothermia and prevent perioperative hypothermia.

Liquid-based patient warming systems generally include a control unit or system that pumps or draws a warming fluid through tubing and into a patient warming device that is in contact with the patient. Traditional connectors used to connect the patient warming device to the control unit are bulky and industrial in nature, and are reusable, rather than disposable (e.g., replaced with each use or after several uses). Generally, separate and distinct connector ports are used for fluid moving from the control unit to the warming device (e.g., outlet line) and from the warming device to the control unit (e.g., inlet line). Thus, connecting and disconnecting the connector ports for each direction of fluid movement requires multiple steps to connect or disconnect connector ports from the control unit and/or the patient warming device. Additionally, care is required to keep the connector ports clean and sterile. Current connectors also do not include built-in features to prevent leakage during disconnection, and typically require a user (e.g., physician or other medical staff or personnel) to actuate a separate slide clamp or tube clamp on both inlet and outlet lines to prevent fluid leakage from the system. Accordingly, there is a need for an improved connector that is more efficient for the user, such as to allow quick connecting and disconnecting of connector ports while preventing leakage from the system and minimizing work involved in maintaining a clean and sterile system for use in medical procedures.

### BRIEF SUMMARY

In one aspect, a connection device for a patient warming system is provided. The device includes a first connector port and a second connector port. The first connector port includes a wall that circumferentially defines a first longitudinal channel. Nearer a first end of the first connector port, the port is configured to receive a first tubing. The second connector port includes a wall that circumferentially defines a second longitudinal channel. Nearer a first end of the second connector port, the port is configured to receive a second tubing. The connection device also includes a first self-sealing valve and a second self-sealing valve. The first valve is aligned within the first longitudinal channel nearer a second end of the first connector port, and the first valve is configured to engage an outlet port of a control unit of the patient warming system. The second self-sealing valve is aligned within the second longitudinal channel nearer a second end of the second connector port, and the second valve configured to engage an inlet port of the control unit of the patient warming system. A planar base member of the connection device is generally perpendicular to the first and second connector ports. The planar base member joins the second end of the first connector port generally parallel to the second end of second connector port. A first flexure member extends from the planar base member towards the first ends of the first and second connector ports. The first flexure member has a first catch mechanism that releasably secures the connection device to the control unit of the patient warming system.

In another aspect, a method is provided for releasably connecting a patient warming device to a control unit in a patient warming system. The method includes connecting a first tubing to a first connector port of a connection device by coupling the first tubing to a first end of the first connector port. The first connector port includes a wall that circumferentially defines a first longitudinal channel. A second tubing is connected to a second connector port of the connection device by coupling the second tubing to a first end of the second connector port. The second connector port includes a wall that circumferentially defines a longitudinal channel. A first valve is aligned in the first longitudinal channel nearer a second end of the first connector port, and the first valve is configured to receive an outlet port of a control unit of the patient warming system. A second valve is aligned in the second longitudinal channel nearer a second end of the second connector port, and the second valve is configured to receive an inlet port of the control unit of the patient warming system. The connection device is inserted into a receiving portion of the control unit of the patient warming device until a shoulder protruding from a first flexure member of the connection device engages a complementary lip on the receiving portion of the control unit to releasably secure the connection device in the receiving portion of the control unit.

Other systems, methods, features, and advantages of the disclosure will be, or will become, apparent to one with skill in the art upon examination of the following figures and detailed description. It is intended that all such additional systems, methods, features and advantages be included within this description, be within the scope of the invention, and be protected by the claims.

### BRIEF DESCRIPTION OF THE DRAWINGS

FIG. 1 is a view of an exemplary control system or control unit for a patient warming system;

FIG. 2 is a first view of an exemplary receiver for a patient warming system;

FIG. 3 is a second view of an exemplary receiver for a patient warming system;

FIG. 4 is a view of an exemplary connection device for a patient warming system;

FIG. 5 is view of an exemplary connection device for a patient warming system with a patient warming device;

FIG. 6 is a view of an exemplary patient warming system connection device;

FIG. 7 is a view of an exemplary connection device for a patient warming system;

FIG. 8A is a view of an exemplary connection device for a patient warming system;

FIG. 8B is a view of an exemplary connection device for a patient warming system with fluid valves;

FIG. 9A is a view of an exemplary patient warming system connection device with supporting ribs;

FIG. 9B is a view of an exemplary patient warming system connection device with supporting ribs and fluid valves;

FIG. 10A is a diagrammatic view of a cross-section of an exemplary patient warming system connection device, taken along line 10A-10A of FIG. 9A;

FIG. 10B is a diagrammatic view of a cross-section of an exemplary patient warming system connection device with fluid valves, taken along line 10B-10B of FIG. 9B;

FIG. 11 is a view of an exemplary patient warming system connection device; and

FIG. 12 is another view of an exemplary patient warming system connection device.

### DETAILED DESCRIPTION

Various embodiments are described below with reference to the drawings in which like elements generally are referred to by like numerals. The relationship and functioning of the various elements of the embodiments may better be understood by reference to the following detailed description. However, embodiments are not limited to those illustrated in the drawings. It should be understood that the drawings are not necessarily to scale, and in certain instances details may have been omitted that are not necessary for an understanding of embodiments disclosed herein, such as—for example—conventional fabrication and assembly. The invention is defined by the claims, may be embodied in many different forms, and should not be construed as limited to the embodiments set forth herein; rather, these embodiments are provided so that this disclosure will be thorough and complete, and will fully convey enabling disclosure to those skilled in the art. As used in this specification and the claims, the singular forms “a,” “an,” and “the” include plural referents unless the context clearly dictates otherwise. Furthermore, use of the terms “first,” “second,” etc., do not denote any order or importance, but rather are used to distinguish between one element and another.

A connection device for a patient warming system and a method for releasably connecting a patient warming device to a control unit in patient warming system are provided in some embodiments. A control system, such as a control unit or control pump, for regulating temperature and a method for regulating temperature in a patient warming system are also provided in some embodiments. The connection device, or connection apparatus, includes a first connector port and a second connector port. The first connector port of the connection device is coupled to a first tubing that leads from a control system, or control unit, of the patient warming system to a patient warming device configured to contact or wrap around at least a portion of a patient's body. A warming fluid flows from the control system, or control unit, through the first connector port and the first tubing into the patient warming device. The second connector port of the connection device is coupled to a second tubing that leads into the control system, or control unit, of the patient warming system from the patient warming device. As used herein, the term “coupled” refers to components that are directly or indirectly connected or attached, and/or permanently or removably connected or attached.

The warming fluid returns from the patient warming device through the second tubing and the second connector port into the control system, or control unit. The control system circulates warming fluid through the patient warming device and controls temperature of the fluid so as to maintain normothermia of the patient or to treat hypothermia. As used herein, normothermia is defined as a range of body core temperature between about 36.5° C. to 37.5° C.±0.5° C. (about 97.7° F. to 99.5° F).±0.9°. Hypothermia is defined as a core temperature less than about 36° C. (about 96.8° F.). Mild hypothermia is defined as ranging from about 1° C. to 2° C. (about 1.8° F. to 3.6° F.) below body core temperature, while moderate hypothermia constitutes a body core temperature of about 35° C. (about 95° F.), and severe hypothermia is a body core temperature below 35° C.

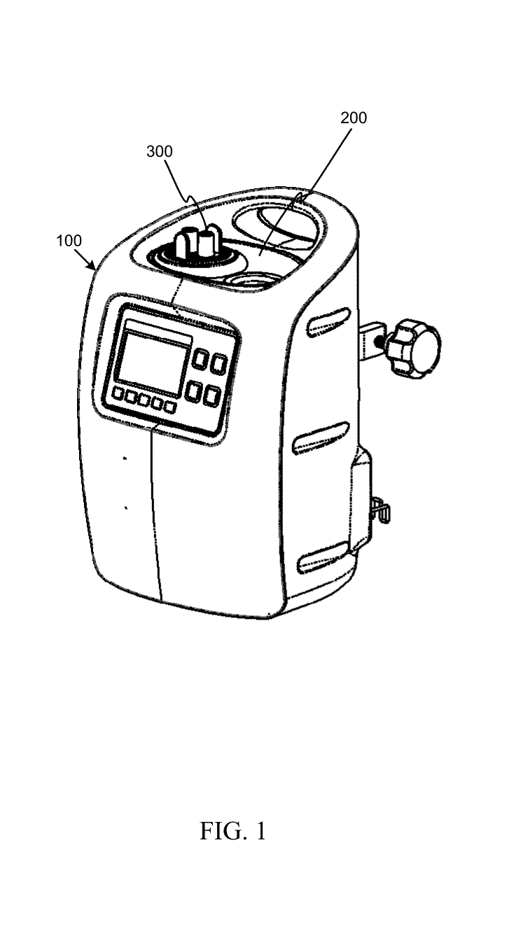

With reference to FIGS. 1-3, an exemplary embodiment of a control unit 100, or control system, includes a receiver 200 with a receiving portion 202 configured (e.g., sized, shaped, and located) to receive a connection device 300, as described below, at the top of the control unit 100. Alternatively the receiver 200 may be located on a side portion of the control unit 100 or a bottom portion of the control unit 100. The receiving portion 202 may include one or more complementary lips 204, 206 that are configured to engage one or more catch mechanisms on the connection device to allow the connection device 300 to be releasably secured in the receiving portion 202 of the control unit 100. The control unit 100 also includes an inlet port 208, through which a warming fluid returns to the control unit 100, and an outlet port 210 through which the warming fluid is delivered from the control unit 100 to a patient warming device 400. Examples of patient warming devices contemplated include those described in U.S. patent application Ser. No. 13/801,270 to Varga, et al.; Ser. No. 13/801,334 to Varga, et al.; and Ser. No. 13/801,512 to Varga, et al., each of which is incorporated herein by reference.

The receiving portion 202 may also include a sensor 212 that detects when the connection device 300 is secured in or removed from the receiving portion 202. The sensor 212 may be activated by pressure, electrical contact, mechanical contact, radio frequency identification (RFID), capacitive sensing, magnetic contact, and/or optical feedback, such as in response to a reflective surface or a scanned barcode, or any combination thereof. The inlet and outlet ports 208, 210 may be inset and surrounded by a wall 214 defining the receiving portion 202. The sensor may be located on a bottom surface 222 of the receiving portion 202, on a wall 214, or at any location on the receiving portion 202 that comes within close proximity or engages a portion of the connection device 300. The warming fluid may be water or other aqueous liquids, a viscous gel, an organic liquid (e.g., oil or oil-based liquid, or any other organic liquid or flowable material with a heat capacity suitable for effective use in keeping with the principles of the present disclosure), a synthetic oil, a foam, or any combination thereof, or any other liquid that has appropriate heat transfer qualities, e.g. high heat capacity and high thermal conductivity, to deliver heat quickly and efficiently to the patient. In some embodiments, the receiver 200 may include a cover 216 that is configured to cover the inlet and outlet ports 208, 210, such as when the control unit 100 is not in use and/or connected to a connection device 300. The cover 216 may prevent unwanted dust, dirt, or other foreign particles or liquids from entering the control unit 100 and may also prevent warming fluid from leaking out of the control unit 100. In some embodiments, the receiver 200 may include a fluid return drain 218 that allows warming fluid to funnel back into the control unit 100, such as when warming fluid escapes from the connection device 300, or otherwise collects in a funneling portion 220 of the receiver 200. For example, the funneling portion 220 may curve or incline inwards to collect fluid and direct fluid into the fluid return drain 218.

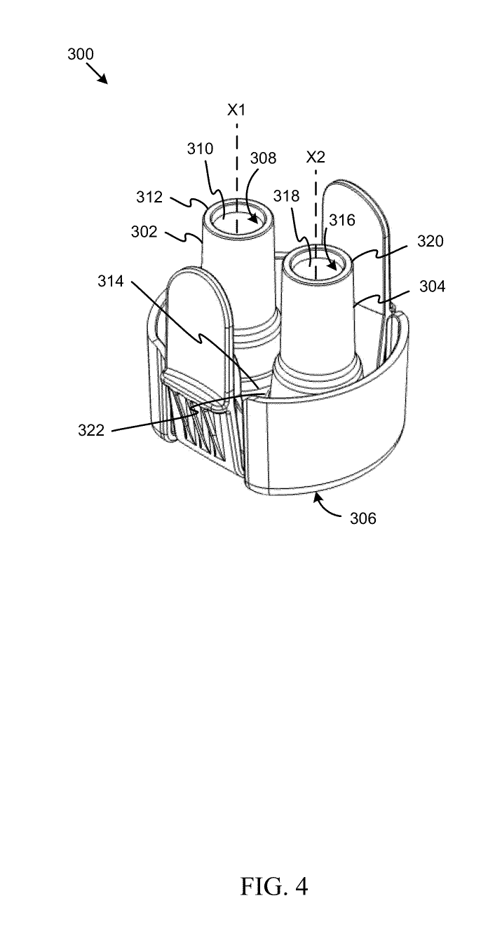

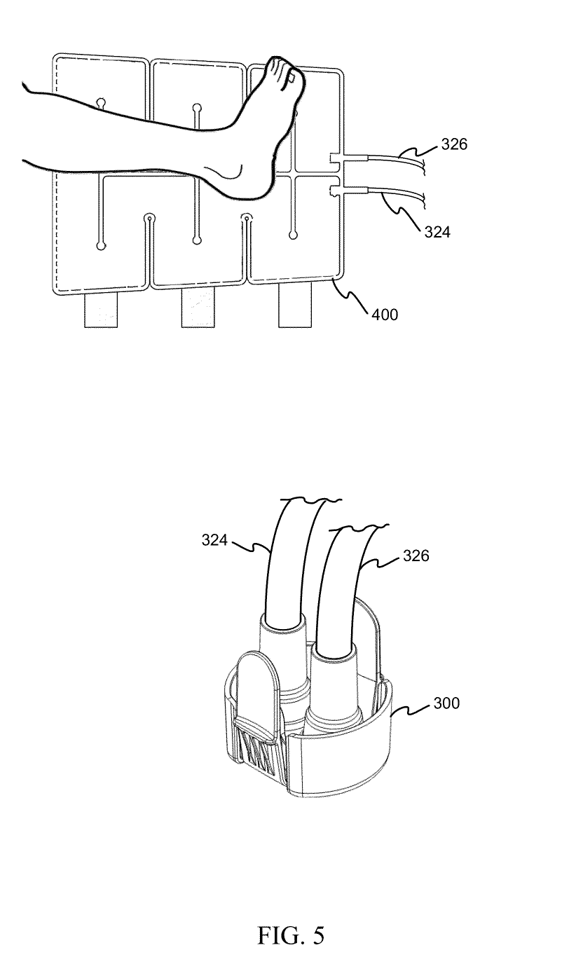

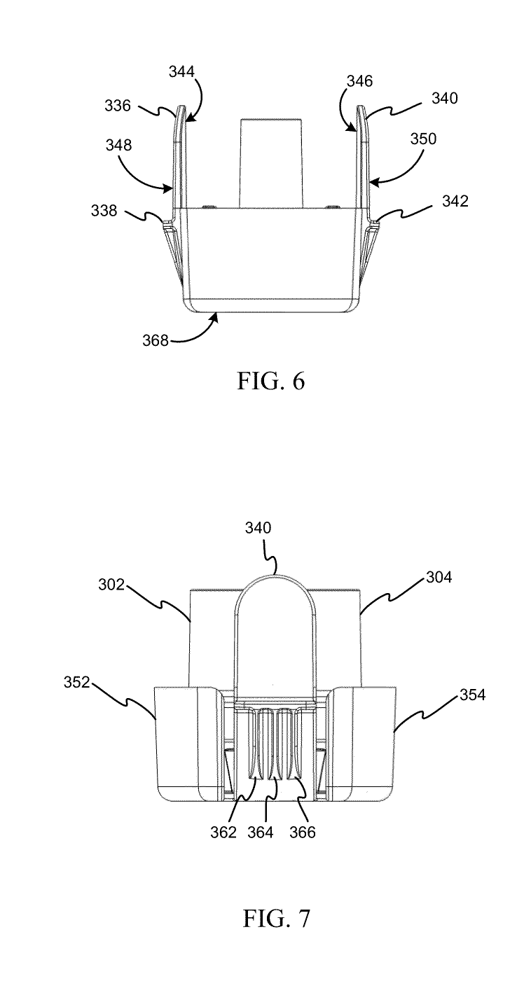

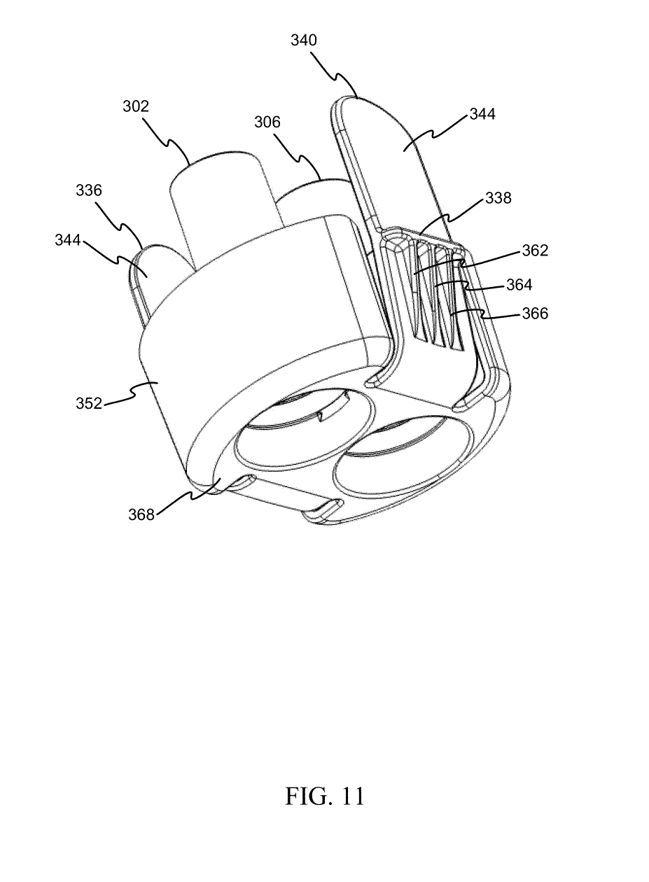

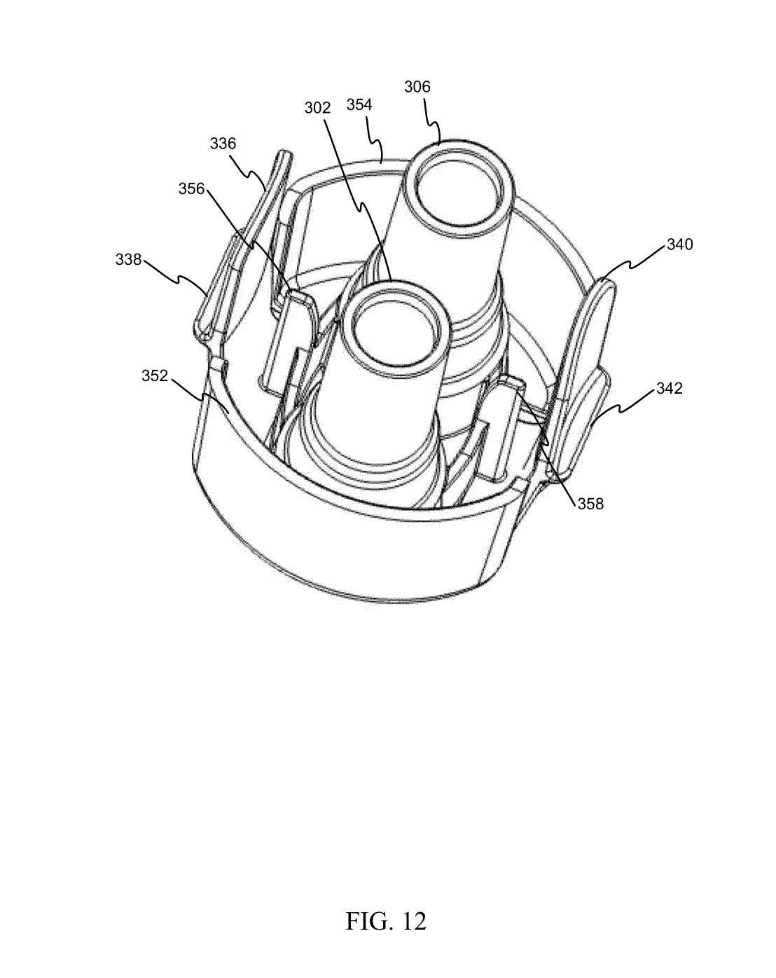

With reference to FIGS.4-12, some embodiments of the connection device 300 include a first connector port 302 and a second connector port 304 joined, or connected, together by a planar base member 306. The first connector port 302 has a wall 308 that circumferentially defines a first longitudinal channel 310 along axis X1. The first connector port 302 also has a first end 312, or a free end, and a second end 314, or a base end, near or at the planar base member 306. The second connector port 304 has a wall 316 that circumferentially defines a second longitudinal channel 318 along axis X2. The second connector port 304 has a first end 320, or free end, and a second end 322, or base end, near or at the planar base member 306. Other shapes are also contemplated for the longitudinal channel, such as oblong, spiraled, rectangular, square, triangular, or other regular or irregular shape. As used herein, the term “circumferentially” does not require a perfect circle and may include, for example, a channel with a generally circular cross-sectional profile, such as one including grooves, dents, or other irregularities in the profile. Also, as used herein, the term “end” refers to a portion that is at or near a distal or proximal portion of a component and is not limited the utmost extremity. The first and second longitudinal channels 310, 318 may have a consistent diameter or cross-section throughout, or a varying diameter or cross-sectional profile.

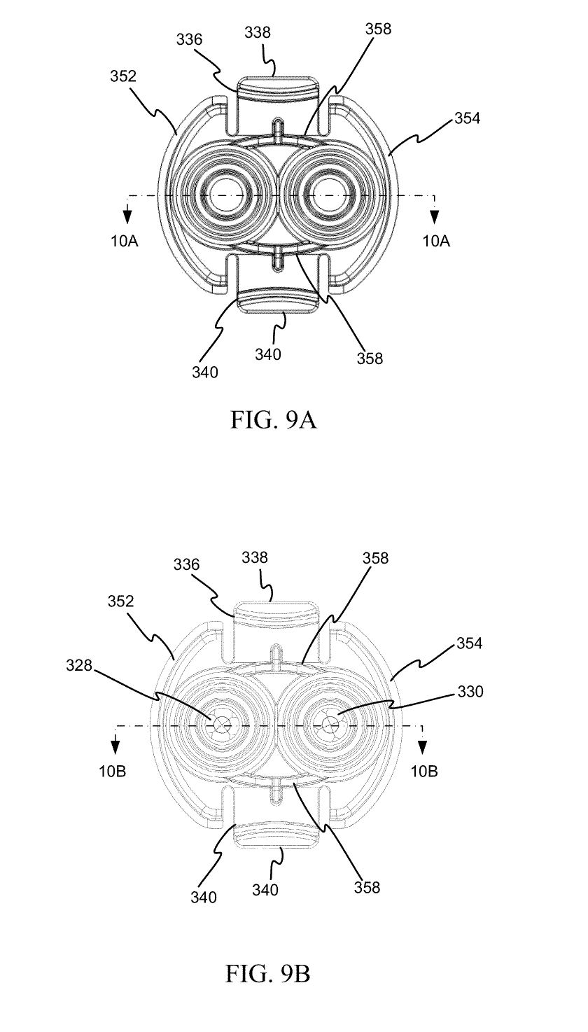

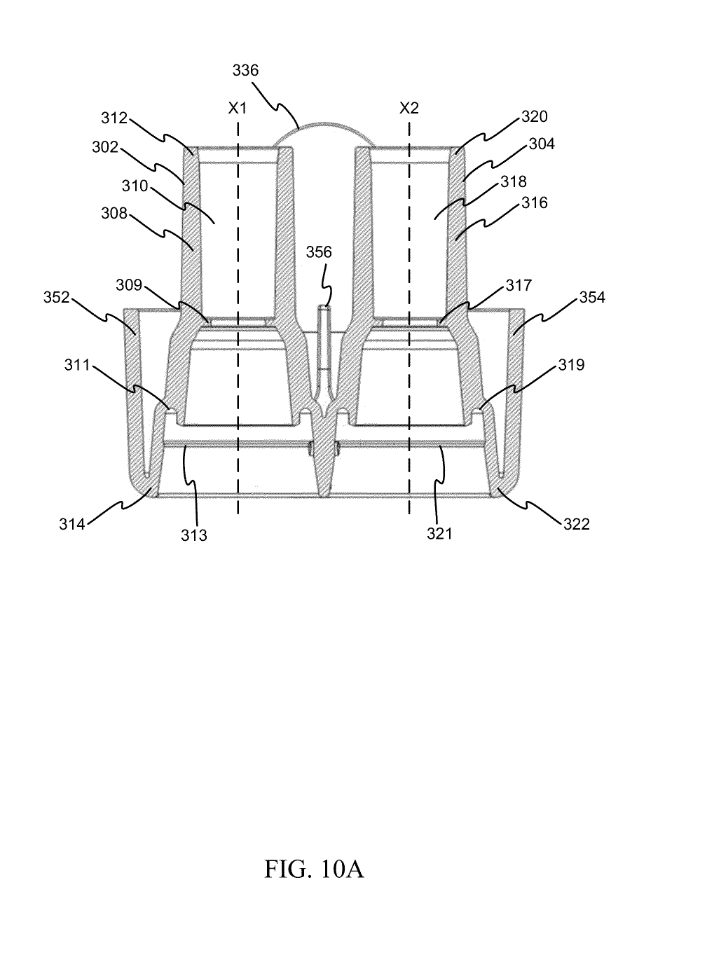

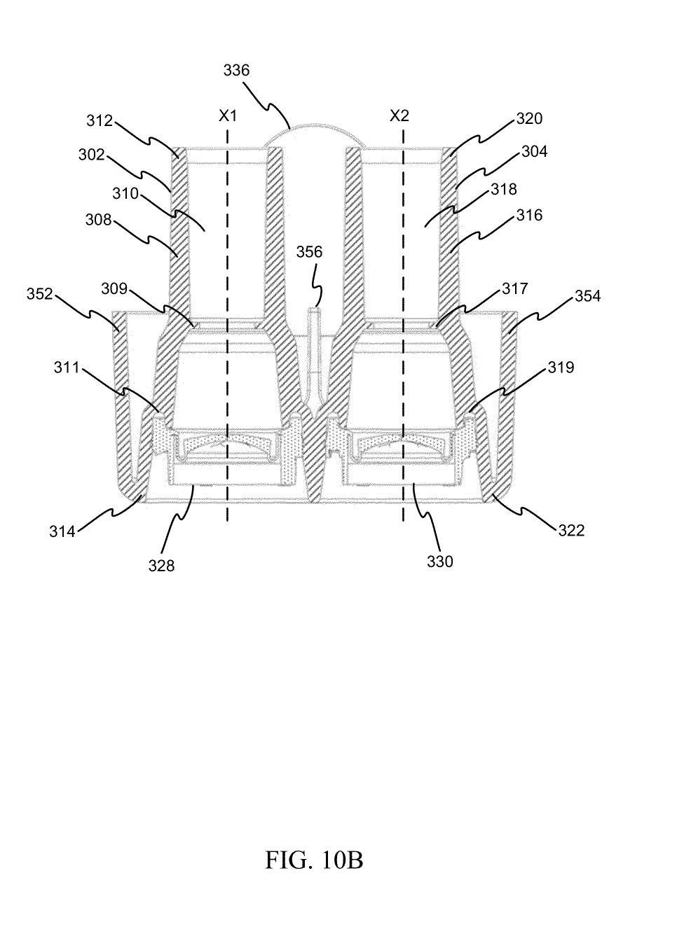

For example, with reference to FIG. 10A, a cross-sectional view taken along line 10A-10A of FIG. 9A is shown, and, with reference to FIG. 10B, a cross-sectional view taken along line 10B-10B of FIG. 9B is shown. The diameter of the longitudinal channels 310, 318 at the first ends 312, 320 may be larger or smaller than the diameter at the second ends 314,322. The diameter or cross-sectional profile of each longitudinal channel 310, 318 may gradually increase, decrease, or otherwise vary from one end to the other, or may vary throughout the length of the channels 310, 318, such as to fit tubing, valves, or other components. Each of these constructions may be useful to create and maintain a fluid connection between the control unit and tubing to the patient warming device and to prevent leaks from the connection device or patient warming system.

The walls 308, 316 may include indents, notches, protrusions, grooves, shoulders, curves or other surface features, or any combination thereof, that are configured for fittings, valves, tubing, to be inserted, connected, or otherwise coupled to the connection device, to secure or maintain components in place, and to maintain or control fluid flow rate. For example, the walls 308, 316 may each include a shoulder 309, 317 that locates the first and second tubing 324, 326 within the connection device 300, such as to limit the length of tubing that is inserted into the longitudinal channels 310, 318. Additionally or alternatively, the walls 308, 316 may each include a grooved shoulder 311, 319 that is configured to fit valves 328, 330, such as to limit the depth at which the valves 328, 330 are located within the second ends 314, 322 of the connector ports 302, 304. Each connector port 302, 304 may also include a valve retention feature 313, 321 (illustrated here as an undercut, but able to be embodied as a different retention feature in other aspects) that holds the valves 328, 330 in place within the longitudinal channels 310, 318, such as by engaging a complementary structure (such as, for example, collar, shoulder, or other structure) on the valves 328, 330 that allow the valves to be secured, for example, by being snapped into place.

The planar base member 306 is generally perpendicular to the first and second connector ports 302, 304. It is also contemplated that in some embodiments, the first and second connector ports 302, 304 are configured at an angle that is not generally perpendicular from the planar base member 306. The first connector port 302 is configured to receive, nearer its first end 312, a first tubing 324. The second connector port 304 is configured to receive, nearer its first end 322, a second tubing 326. The first and second tubing 324, 326 lead to a patient warming device 400, such as a wrap that surrounds and conforms to a body portion, appendage, or extremity of a patient so as to maintain normothermia of the patient or to treat hypothermia. The connection device forms a user-actuatable, and easily inter-changeable, fluid-patent connection between the tubing and the control unit, so as to allow the control unit to circulate/direct warming fluid through the first tubing 324 to the patient warming device 400. The second tubing 326 carries fluid from the patient warming device 400 back to the control unit. Alternatively, the control unit may circulate/direct warming fluid through the second tubing 326 to the patient warming device 400, and the first tubing 324 may carry the fluid from the patient warming device 400 back to the control unit.

The first and second connector ports 302, 304 and the planar base member 306 may be formed as a single component, such as through a plastic molding process. Alternatively, the first and second connector ports 302, 304 and the planar base member 306 may be separate components that are assembled together to form the connection device 300 to form leak proof joints, such as being sealed together by a solvent or adhesive. Appropriate materials for the connection device 300 include PVC, acrylic, polypropylene, ABS, polycarbonate, nylon, PET and PBT polymers, or any combination thereof, and/or other materials that are sufficiently rigid to maintain positioning of the connector ports so as to allow proper alignment with the inlet and outlet ports of the receiving portion 202 of the control unit when the connection device 300 is secured to the receiving portion 202. The connector ports and the planar base member may be made of the same material or different materials. In some embodiments, the connection device 300 may include any number of connector ports, and the receiving portion 202 of the control unit 100 may include any number of inlet and outlet ports. The control unit 100 may also include any number of receiving portions, such as to receive multiple connection devices 300 connected to deliver or circulate warming fluid to one or more patient warming devices. Alternatively or additionally, in some embodiments, a patient warming system may include additional tubing, or lines, to transmit gas (e.g., air). For example, the control unit 100 and the connection device 300 may include mating ports configured to transmit gas to and from components of the warming device 400. Such tubing or lines are not shown in the present drawings, but one of skill in the art will be able (with reference to the teachings of the present disclosure) to envision and construct the structures described here.

The diameter, length, thickness and material of the tubing may be configured to control the amount of heat loss to the ambient environment as fluid travels through the first and second tubing 324, 326 between the control unit and the patient warming device 400. The first and second tubing 324, 326 may share the same diameter, length, thickness and material, or may differ in any or all of these properties. Depending on the application, the length of the tubing, as measured from the connection device 302 to the patient warming device 400, may vary from about five feet to about 12 feet long. The first and second tubing 324, 326 are made of flexible material, such as PVC (polyvinyl chloride), silicone, urethane, polyurethane, PE, EVA, EVA/PE blends or copolymers, SBC, medical elastomers, olefin-based compounds, or other material suitable for use in sterile environments. The thickness and/or cross-sectional area or profile of the tubing may be configured to the reduce likelihood of kinking, for example, during placement of the patient warming device 400 on the patient while the connection device 300 is secured to the control unit 100. Material for the tubing may have insulating properties, or an insulating sleeve may surround the tubing, to reduce the amount of heat loss to the environment while the warming fluid travels through the tubing. The tubing may be inserted into the longitudinal channels of the connector ports so as to form a leak resistant seal. Alternatively, the first ends of the connector ports may be sized to fit in the inner diameter of the tubing and to form a leak resistant seal. In some embodiments, the tubing may be sealed to the connection ports by a solvent to create a leak proof seal. Alternatively, tubing may be welded to the connection ports by a plastic welding technique.

The connection device further includes a first valve 328 aligned in the first longitudinal channel 310 nearer the second end 314 of the first connector port 302 and a second valve 330 aligned in the second longitudinal channel 318 nearer the second end 322 of the second connector port 304. The first valve 328 is configured to engage an outlet port 210 of the control unit, and the second valve 330 is configured to engage an inlet port 208 of the control unit. The inlet and outlet ports 208, 210 may be inset in the receiving portion 202 and surrounded by a wall 214 defining the receiving portion 202. The depth of the receiving portion 202 may correlate with or match exactly (or nearly exactly) the height of at least a main body portion defined by the height(s) of the sidewalls 352, 354 of the connection device 300 (excepting the tabs 336, 340, which extend above that receiving portion depth). In certain preferred embodiments, the height of the connection device or a predetermined portion thereof will match exactly or nearly exactly the depth of the receiving portion so that the connected fit therebetween presents an aesthetic appearance of good fit.

In some embodiments, the connection device 300, the first and second tubing 324, 326, and the patient warming device 400 are provided as a pre-packaged assembly that can be quickly attached to the control unit by securing the connection device 300 to the receiving portion 202 of the control unit 100. For example, when the connection device 300 is secured to, or inserted into, the receiving portion 202, the outlet port 210 fits securely into the first valve 328 of the first connector port 302 and the inlet port 208 fits securely into the second valve 330 in the second connector port 304. In operation, the control unit 100 pumps the warming fluid through the outlet port 210 past the first valve 328 and first connector port 302, through the first tubing 324, and into the patient warming device 400. As the control unit 100 continues to operate, the patient warming device 400 fills and fluid is circulated through the patient warming device 400 and into the second tubing 326, and back into the control unit 202 through the second connector port 304 and the inlet port 208 past the second valve 330. Additionally or alternatively, the connection device 300, the first and second tubing 324, 326, and the patient warming device 400 may be provided as individual components, or in any combination of components, such as for replacement parts or customizable configurations.

The first and second valves 328, 330 may be constructed as self-sealing valves that substantially prevent leakage when the connection device 300 is released (e.g., removed or detached) from the receiving portion 202 of the control unit. For example, the valves 328, 330 may be silicone rubber, or other suitable flexible material that is strong enough to prevent fluid from escaping through slits radiating from a center of the valve. The slits allow a tubing or connector to be inserted into the valve and the flexible material of the valve conforms to the shape of the tubing or connector to prevent leakage while the tubing or connector is inserted through the valve. When the tubing or connector is withdrawn from the valve, the flexible material returns to a closed position in which no fluid escapes from the slits. Examples of valve configurations contemplated include those described in U.S. Pat. No. 6,293,437 to Socier, et al.; U.S. Pat. No. 6,405,901 to Schantz, et al.; and U.S. Pat. No. 7,980,430 to Hickok, et al., each of which is incorporated herein by reference.

Releasing the connection device 300 from the control unit 100, such as by squeezing the flexure tabs 336, 340 and lifting the connection device away from the control unit disconnects both the first and second tubing 324, 326. This may enable disconnection of the patient warming device 400 from the control unit 100 without separately clamping each tube and without requiring disconnection of a separate connector for each tube. The fluid that is in the patient warming device 400 and the first and second tubing 324, 326 is maintained therein by the first and second valves 328, 330. Medical personnel may then discard the pre-packaged assembly, including the warming fluid that fills it, after use. Alternatively, the pre-packaged assembly may be provided pre-filled with warming fluid that can be circulated into the control unit when the pump of the control unit is activated. During operation of the control unit (e.g., when fluid is being circulated from the control unit to the patient warming device 400 and back into the control unit), the first and second valves 328, 330 also form substantially leak proof seals between the first connector port 302 and the inlet port 208 and between the second connector port 304 and the outlet port 210. The control unit may continuously circulate or pump the warming fluid into the patient warming device 400 and back into the control unit. Alternatively or additionally, the control unit may pump the warming fluid into the patient warming device 400 at regular pulsatile intervals, or at varying intervals to accommodate for desired flow rate and heat exchange.

In some embodiments, the connection device 300 includes a first flexure member 336 that extends from the planar base member 306 towards the first ends 312, 320 of the first and second connector ports 302, 304. The first flexure member 336 has a first catch mechanism 338 that is configured to releasably secure the connection device 300 to the control unit of the patient warming system. The connection device 300 may further include a second flexure member 340 that extends from the planar base member 306 towards the first ends 312, 320 of the first and second connector ports 302, 304. The second flexure member 340 includes a second catch mechanism 342 configured to releasably secure the connection device to the control unit of the patient warming system. The first and second flexure members 336, 340 may be flexible tabs that each have an internal surface 344, 346 that faces the first and second connector ports 302, 304. Alternatively or additionally, the connection device 300 may include any number of one or more flexure members.

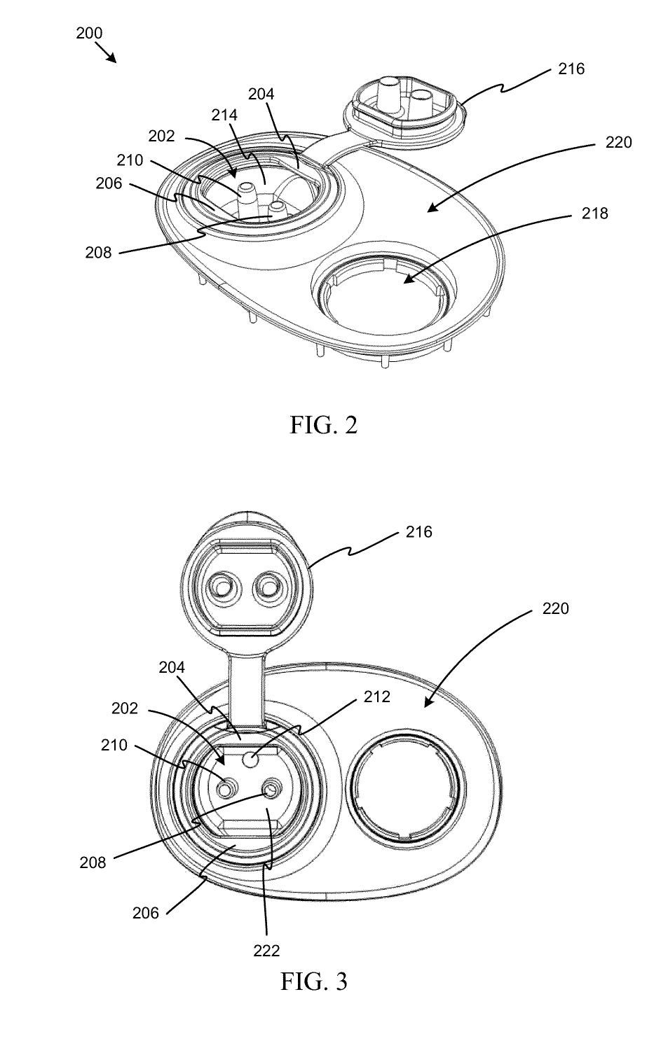

With reference to FIGS. 2-9B, in an exemplary embodiment of the connection device 300, the first and second catch mechanisms 338, 342 are each a shoulder protruding from an external surface 348, 350 of the first and second flexure members 336, 340. A receiving portion 202 on the control unit includes one or more complementary lips 204, 206. When the connection device 300 is secured to (e.g., aligned with or inserted into) the receiving portion 202 of the control unit, the first and second catch mechanisms 338, 342 are configured to engage the complementary lips 204, 206. When the first and second flexure members 336, 340 flex inwards, such as when a user squeezes the flexure members inwards, towards the first and second connector ports 302, 304, the first and second catch mechanisms are released from engagement with the lip on the control unit. The complementary lip on the control unit may be a continuous protrusion along an edge of the receiving portion 202 of the control unit, or the complementary lip may include one or more protrusions that are configured (e.g., sized, shaped, and located) to engage the catch mechanisms on the flexure members. Alternatively or additionally, the catch mechanisms may be any type of protrusion or structure that engages with a complementary mechanism of the receiving portion 202. For example, the catch mechanisms may be rounded protrusions, or balls, and the complementary mechanism may be a dent, such that the rounded protrusion catches on the dent as in a ball and dent connector.

As another example, the catch mechanism may snap fit into the complementary mechanism. Appropriate materials for the receiving portion 202 may include PVC, acrylic, polypropylene, ABS, polycarbonate, nylon, PET and PBT polymers, any combination thereof, and/or any other material sufficiently rigid to reduce the likelihood of the inlet and outlet ports 208, 210 from being displaced, such as to cause a leak in the system. The lips 204, 206 and/or other parts of the receiving portion 202 may be made of metal, and/or other material that is more rigid than other areas, in order to resist wear from large numbers of connectors 300 being connected and disconnected over the useful life of the controller 100.

In some embodiments, the connection device 300 also includes a first sidewall 352 and a second sidewall 354 opposite the first sidewall 352. Each of the first and second sidewalls 352, 354 extend from the planar base member 306 towards the first ends 312, 320 of the first and second connector ports 302, 304. For example, as shown in FIG. 9, the first and second sidewalls 352, 354 and the first and second flexure members 336, 340 together generally surround the first and second connector ports 302, 304. The first and second sidewalls 352, 354 and the first and second flexure members 336, 340 form a continuous wall around the first and second connector ports, such as along the edge or near the edge of the planar base member 306. Alternatively or additionally, the flexure members 336, 340 may be formed as individual tabs extending from the planar base member 306, or may be defined by slits or cutouts (e.g., extending generally or substantially perpendicular to the planar base member 306) in the first and second sidewalls 352, 354. The first and second sidewalls 352, 354 and the first and second flexure members 336, 340 may be separate components that are attached to the edges or near the edges of planar base member 306, or may be formed (e.g., through a plastic molding process) as a continuous component with the planar base member 306. The connection device 300 may include any number of sidewalls. The sidewalls may be configured to increase rigidity of the planar base member 306 and/or the connection device 300 as a whole.

With reference to FIGS. 9A-9B, and 10A-10B, for example, some embodiments of the connection device 300 include supporting ribs 356, 358 that extend from the planar base member 306 generally parallel to the axes X1 and X2 of the first and second longitudinal channels 310, 318. The supporting ribs 356, 358 may extend fully or partially between the first and second ports 302, 304, for example, to increase rigidity of the planar base member 306 and/or the connection device 300 as a whole. With reference to FIG. 12, for example, the ribs 356, 358 may be located between the flexure members 336, 340 and the connector ports 302, 304. The supporting ribs 356, 358 may have a uniform profile throughout, or may vary, such as to include a portion that extends from the planar base member 306 towards the first ends 312, 320 of the connector ports 302, 304, such as to prevent the flexure members 336, 340 from flexing inward to contact the connector ports 302, 304. Preventing the flexure members 336, 340 from contacting the connector ports 302, 304 is desirable, for example, to avoid unintentional displacement of the first and second tubing 324, 326. The connection device 300 may include any number of one or more supporting ribs extending from the planar base member 306.

In some embodiments, the connection device 300 includes supporting ridges 362, 364, 366 on one or more of the flexure members 336, 340. The supporting ridges 362, 364, 366 may extend or protrude from the external surface 348, 350 of each flexure member 336, 340 to form all or a part of each catching mechanism 338, 342. Alternatively, the catching mechanisms 338, 342 may be formed as a simple lip, or other protrusion with a simple profile that does not include supporting ridges. In some embodiments, the catching mechanisms 338, 342 are located near a central portion of each flexure member 336, 340, at a height that allows an external bottom surface 368 of the connection device 300 to meet a contact surface 222 and/or the sensor 212 of the receiving portion 202 while engaging the complementary lips 204, 206 of the receiving portion 202. For example, the distance between the bottom surface 368 of the connection device 300 and the end of the catching mechanism is substantially equal to the distance between the contact surface 222 and the complementary lips 204, 206. Maintaining a small clearance or zero clearance between the connection device 300 and the receiving portion 202 may help avoid leaks and dislocation when the connection device 300 is secured to the receiving portion 202. Additionally or alternatively, maintaining a small clearance or zero clearance may help to avoid spurious disconnection signals by the sensor 212. In some embodiments, the supporting ridges 362, 364, 366 extend fully or partially along the distance between the ends of the catching mechanisms 338, 342 to the bottom surface 368, such as, for example, to provide additional rigidity to a portion of the flexure members 336, 340 while allowing flexibility in other portions of the flexure members 366, 340. Some flexibility in the flexure members 366, 340 may be desirable to allow release of the catching mechanisms 338, 342 when pressure is applied to the flexure members (e.g., when the flexure members are squeezed in towards the first and second ports 302, 304).

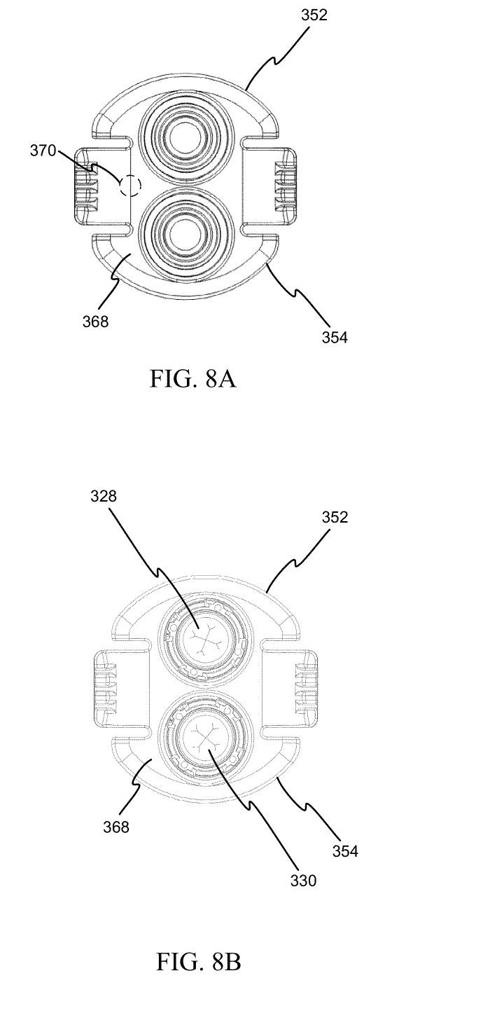

With reference to FIGS. 8A and 8B, for example, some embodiments of a connection device include a bottom surface 368 with a communication target, such as a contact point, proximity target, or other surface feature 370 that enables detection of proximity between the connection device 300 and the receiving portion 202 of the control unit. For example, detection of proximity is enabled by pressure, electrical contact, mechanical contact, radio frequency identification (e.g., RFID chip/sensor), capacitive sensing, magnetic contact, and/or optical feedback, such as in response to a reflective surface or a scanned barcode, or any combination thereof, between the target or surface feature on the bottom surface 368 and the sensor 212 of the receiving portion 202 of the control unit. The information sensed and/or communicated may include such data as the volume of the patient warming device, target temperature ranges, flow-rates, and/or any other relevant data by which the control unit could operate in a manner particularly suited for a given patient warming device. When the connection device is properly secured to the control unit, such as when the connection device is inserted into the receiving portion and the bottom surface of the connection device is sufficiently close in proximity to the bottom of the receiving portion, the sensor 212 and/or target 370 may trigger an alert and/or notification to the system, control unit, or user that the connection device is properly secured. Alternatively or additionally, a communication target may be located on any other portion of the connection device, such as on a sidewall or flexure tab.

Increased rigidity of a connection device is desirable, for example, to prevent or reduce dislocation or movement of the connector ports. For example, when the flexure members are activated (e.g., squeezed) to release the connection device 300, or when the connection device 300 is being secured to, the receiving portion 202 of the control unit, movement or dislocation of the connector ports during secure and release of the connection device may cause leakage as the inlet and outlet ports 208, 210 are inserted into or withdrawn from the connector ports 302, 304

In some embodiments, the control unit 100 pumps the warming fluid at a rate of at least about 500 mL/min (30.5 in³/min) to about 800 mL/min (48.8 in³/min) to deliver sufficient heat to maintain normothermia or treat hypothermia of the patient during functional use of the patient warming device 400. To increase heat delivery to the patient, the flow rate may be increased to about 2 L/min (0.07 ft³/min) or more. The desired flow rate may be adjusted by using the control unit 100. The control unit 100 circulates the warming fluid to the patient warming device 400, which delivers heat to the patient across the surface area of the patient warming device 400. The warming fluid may include, for example, a warm liquid, such as water or other aqueous liquids, a viscous gel, a hydrogel, an organic liquid (e.g., oil or oil-based liquid, or any other organic liquid or flowable material with a heat capacity suitable for effective use in keeping with the principles of the present disclosure), a synthetic oil, a foam, or forced air, or any combination thereof. The warming fluid is cooled as heat is delivered to the patient, and pumped out of the patient warming device 400 by the control unit 100. Alternatively, or additionally, the cooled fluid may exit the patient warming device 400 through another mechanism, such as a vacuum, suction, or drain.

In some embodiments, a patient warming system may include sensors to monitor temperature and pressure applied at the surface of the appendage or body portion. For example, sensors may be placed on the underlying surface of the patient body portion or appendage to be surrounded by the patient warming device 400 or may be incorporated into the patient warming device 400. The sensors may be coupled to a system controller, such as the control unit 100. As the fluid fills the device, the sensors may provide a feedback signal to the fluid control unit 100, which may be configured to adjust the temperature or the flow rate of the fluid entering the warming device 400 so as to maintain the temperature of the underlying surface within a predetermined range to maintain normothermia. For example, the temperature or flow rate of the fluid may be adjusted to maintain a temperature at the heat transfer surface of between about 36° C. (96.8° F.) and 40° C. (104° F.). Alternatively, or in addition, the heat may be adjusted using the feedback system and patient core temperature monitoring.

Although various embodiments of the invention have been described, it will be apparent to those of ordinary skill in the art that many more embodiments and implementations are possible that are within the scope of the invention. For instance, steps of a method as displayed in the figures or reflected in the claims do not require a specific order of execution by way they are presented, unless specified. The disclosed steps are listed as exemplary such that additional or different steps may be executed or the steps may be executed in a different order. Those of skill in the art will appreciate that embodiments not expressly illustrated herein may be practiced within the scope of the claims, including that features described herein for different embodiments may be combined with each other and/or with currently-known or future-developed technologies while remaining within the scope of the claims.

Those of skill in the art will appreciate that embodiments not expressly illustrated herein may be practiced within the scope of the claims, including that features described herein for different embodiments may be combined with each other and/or with currently-known or future-developed technologies while remaining within the scope of the claims. Although specific terms are employed herein, they are used in a generic and descriptive sense only and not for purposes of limitation unless specifically defined by context, usage, or other explicit designation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment. In the event of any inconsistent disclosure or definition from the present application conflicting with any document incorporated by reference, the disclosure or definition herein shall be deemed to prevail.

We claim:

1. A connection device for a patient warming system, the connection device comprising:
a first connector port comprising a wall that circumferentially defines a first longitudinal channel, the first connector port configured to receive, nearer a first end of the first connector port, a first tubing;
a second connector port comprising a wall that circumferentially defines a second longitudinal channel, the second connector port configured to receive, nearer a first end of the second connector port, a second tubing;
a first self-sealing valve aligned within the first longitudinal channel nearer a second end of the first connector port, the first valve configured to engage an outlet port of a control unit of a patient warming system;
a second self-sealing valve aligned within the second longitudinal channel nearer a second end of the second connector port, the second valve configured to engage an inlet port of the control unit of the patient warming system;
a planar base member, generally perpendicular to the first and second connector ports, that joins the second end of the first connector port generally parallel to the second end of second connector port; and
a first flexure member extending from the planar base member towards the first ends of the first and second connector ports, the first flexure member comprising a first catch mechanism configured to releasably secure the connection device to the control unit of the patient warming system.

2. The connection device of claim 1, further comprising:
a first sidewall and a second sidewall opposite the first sidewall, each of the first sidewall and the second sidewall extending from the planar base member towards the first ends of the first and second connector ports; and
a second flexure member opposite the first flexure member and extending from the planar base member towards the first ends of the first and second connector ports, the second flexure member comprising a second catch mechanism configured to releasably secure the connection device to the control unit of the patient warming system;
wherein the first and second sidewalls and the first and second flexure members together generally surround the first and second connector ports.

3. The connection device of claim 2, wherein:
the first and second flexure members are flexible tabs each comprising an internal surface that faces the first and second connector ports, and
the first and second catch mechanisms are each a shoulder protruding from an external surface of the first and second flexure members and configured to engage a complementary lip on the control unit; and wherein when the first and second flexure members flex inwards towards the first and second connector ports, the first and second catch mechanisms are released from engagement with the lip on the control unit.

4. The connection device of claim 1, further comprising a rib that is attached at its ends to the first and second connector ports and that extends from the planar base member towards the first ends of the first and second connector ports and increases rigidity of the connection device.

5. The connection device of claim 4, wherein the rib includes at least one tab that limits movement of the first and second flexure members so as to prevent the first and second flexure members from contacting the first and second connector ports when the first and second flexure members flex inwards toward the first and second connector ports.

6. The connection device of claim 1, wherein the first longitudinal channel is substantially parallel to the second longitudinal channel.

7. The connection device of claim 1, wherein the first and second flexure members each comprise a cambered contact surface.

8. The connection device of claim 1, wherein the first connector port acts as a female connector and the first tubing is received in the first longitudinal channel.

9. The connection device of claim 1, wherein the first connector acts as a male connector and the first tubing is received around an outer diameter of the first connector.

10. The connection device of claim 1, wherein when the connection device is released from the control unit, the first and second valves are configured to prevent leakage from the first and second tubing.

11. The connection device of claim 1, wherein the first connector port is configured to be coupled to the first tubing to receive a warming fluid from the outlet port of the control unit and allow the warming fluid to flow to a patient warming device.

12. The connection device of claim 1, wherein the second connector port is configured to be coupled to the second tubing to receive a warming fluid from a patient warming device and allow the warming fluid to flow into the inlet port of the control unit.

13. The connection device of claim 1, wherein the planar base member further comprises an external bottom surface including a communication target that, when then the connection device is properly secured to the control unit, communicates to the control unit that the connection device is properly secured.

14. A method for releasably connecting a patient warming device to a control unit in a patient warming system, the method comprising:
connecting a first tubing to a first connector port of a connection device by coupling the first tubing to a first end of the first connector port, the first connector port comprising a wall that circumferentially defines a first longitudinal channel;
connecting a second tubing to a second connector port of the connection device by coupling the second tubing to a first end of the second connector port, the second connector port comprising a wall that circumferentially defines a second longitudinal channel;
aligning a first valve in the first longitudinal channel nearer a second end of the first connector port, wherein the first valve is configured to receive an outlet port of a control unit of the patient warming system;
aligning a second valve in the second longitudinal channel nearer a second end of the second connector port, wherein the second valve is configured to receive an inlet port of the control unit of the patient warming system; and
inserting the connection device into a receiving portion of the control unit of the patient warming device until a shoulder protruding from a first flexure member of the connection device engages a complementary lip on the receiving portion of the control unit to releasably secure the connection device in the receiving portion of the control unit.

15. The method of claim 14, wherein inserting the connection device into the receiving portion of the control unit further comprises:
sliding a first sidewall and a second sidewall opposite the first sidewall of the connection device along a direction of insertion into the receiving portion of the control unit, wherein each of the first sidewall and the second sidewall extends from the planar base member towards the first ends of the first and second connector ports;
flexing the first flexure member inwards towards the first and second connector ports to allow the shoulder protruding from the first flexure member to pass the complementary lip as the connection device is moved in the direction of insertion;
flexing a second flexure member inwards towards the first and second connector ports, wherein the second flexure member is opposite the first flexure member and extends from the planar base member towards the first ends of the first and second connector ports, the second flexure member further comprising an other shoulder configured to engage an other complementary lip on the receiving portion of the control unit; wherein the first and second sidewalls and the first and second flexure members together generally surround the first and second connector ports and provide rigidity to the connection device.

16. The method of claim 15, further comprising squeezing the first and second flexure members inwards towards each other and pulling the connection device in a direction opposite the direction of insertion until the shoulder of the first flexure member and the other shoulder of the second flexure member slide past the complementary lip and other complementary lip of the receiving portion to allow the connection device to be released from the receiving portion of the control unit.

17. The method of claim 14, wherein coupling the first tubing to the first end of the first connector port comprises: inserting the first end of the connector port into the first tubing or inserting the first tubing into the first end of the first connector port.

18. The method of claim 14, further comprising:
inserting the connection device into the receiving portion of the control unit until an external bottom surface of the planar base member; and
when a communication target on the external bottom surface reaches a threshold proximity with a sensor of the receiving portion, communicating to the control unit that the connection device is properly secured to the control unit.

19. The method of claim 14, wherein coupling the first tubing to the first end of the first connector port and coupling the second tubing to the first end of the second connector port creates a sealed fluid system comprising the connection device, the first and second tubing, and a patient warming device in fluid connection with the first and second tubing.

20. The method of claim 19, wherein aligning the first valve in the first longitudinal channel nearer the second end of the first connector port comprises inserting a first self-sealing valve into the first longitudinal channel to prevent a warming fluid from flowing out of the sealed fluid system through the first self-sealing valve, and aligning the second valve in the second longitudinal channel nearer the second end of the second connector port comprises inserting a second self-sealing valve into the second longitudinal channel to prevent a warming fluid from flowing out of the sealed fluid system through the second self-sealing valve.
